SEQUENCE LISTING (13 sequences)

SEQ ID NO: 1 - DNA, 135 bp, homo sapiens
  CDS (3)..(134)
gcttcagcactggggcctatagttcagacagctgttcagcggaagagggtttgcactgcaggcccctctcttgcagaagtggaggtgaggcttgtctctgcttcaaggactctctctgcaacctccaacaggaag

SEQ ID NO: 2 - protein, 44 aa, homo sapiens
FSTGAYSSDSCSAEEGLHCRPLSCRSGGEACLCFKDSLCNLQQE

SEQ ID NO: 3 - DNA, 135 bp, Homo sapiens; identical to SEQ ID NO: 1

SEQ ID NO: 4 - protein, 43 aa, Homo sapiens
FSTGAYSSDSCSAEEGLHCRPLSCRSGGEACLCFKDSLCLQQE

SEQ ID NO: 5 - protein, 363 aa, Homo sapiens
GRNKMDPPRSSIFLQEVITTVYGYKVQLQAGLELVVNNQKMAVPYRPNEHLRVTLRGQRLYLVTDFELVVSFGGRKNAVISLPSMYEGLVSGLCGNYDKNRKNDMMLPSGALTQNLNTFGNSWEVKTEDALLRFPRAIPAEEEGQGAELGLRTGLQVSECSPEQLASNSTQACRVLADPQAPFAACHQTVPPEPFQEHCVLDLCSAQDPREQEELRCQVLSGYAILCQEAGAALAGWRDRTLCAMECPAGTIYQSCMTPCPASCANLADPGDCEGPCVEGCADPRLCLQWHPEPPLADCGCTSNGIYYQVRAGSRRPWGAEAPRRARPGMELERLLLALPFLAGQQFLTEDCSQRCTCASSAI

SEQ ID NO: 6 - DNA, 15 bp, artificial sequence
  primer for PCR amplification used in different display RT-PCR in Example 1.
tgcttcagcactgcc

SEQ ID NO: 7 - DNA, 18 bp, artificial sequence for PCR amplification used in different display RT- PCR in Example  1.
  primer
ttattgtatttgaagtaa

SEQ ID NO: 8 - DNA, 825 bp, Homo sapiens
  CDS (1)..(822)
atggaccggccggggttcgtggcagcgctggtggctggtggggtagcaggtgtttctgttgacttgatattatttcctctggataccattaaaaccaggctgcagagtccccaaggatttaataaggctggtggttttcatggaatatatgctggcgttccttctgctgctattggatcctttcctaatgctgctgcattttttatcacctatgaatatgtgaagtggtttttgcatgctgattcatcttcatatttgacacctatgaaacatatgttggctgcctctgctggagaagtggttgcctgcctgattcgagttccatctgaagtggttaagcagagggcacaggtatctgcttctacaagaacatttcagattttctctaacatcttatatgaagagggtatccaagggttgtatcgaggctataaaagcacagttttaagagagattcctttttctttggtccagtttcccttatgggagtccttaaaagccctctggtcctggaggcaggatcatgtggtggattcttggcagtcagcagtctgtggagcttttgcaggtggatttgccgctgcagtcaccacccctctagacgtggcaaagacaagaattacgctggcaaaggctggctccagcactgctgatgggtatgtgctctctgtcctgcatggggtctggcggtcacaggggctggcaggattatttgcaggtgtcttccctcgaatggcagccatcagtctgggaggtttcatctttctgggggcttatgaccgaacgcacagcttgctgttggaagttggcagaaagagtccttga

SEQ ID NO: 9 - protein, 274 aa, Homo sapiens
MDRPGFVAALVAGGVAGVSVDLILFPLDTIKTRLQSPQGFNKAGGFHGIYAGVPSAAIGSFPNAAAFFITYEYVKWFLHADSSSYLTPMKHMLAASAGEVVACLIRVPSEVVKQRAQVSASTRTFQIFSNILYEEGIQGLYRGYKSTVLREIPFSLVQFPLWESLKALWSWRQDHVVDSWQSAVCGAFAGGFAAAVTTPLDVAKTRITLAKAGSSTADGYVLSVLHGVWRSQGLAGLFAGVFPRMAAISLGGFIFLGAYDRTHSLLLEVGRKSP

SEQ ID NO: 10 - protein, 14 aa, Homo sapiens
KTRLQSPQGFNKAG

SEQ ID NO: 11 - protein, 11 aa, Homo sapiens
GVPSAAIGSFP

SEQ ID NO: 12 - protein, 12 aa, Homo sapiens
SEVVKQRAQVSA

SEQ ID NO: 13 - protein, 39 aa, Homo sapiens
VCGAFAGGFAAAVTTPLDVAKTRITLAKAGSSTADGYVL

(19) United States
(12) Patent Application Publication
(10) Pub. No.: US 2003/0219424 A1
(43) Pub. Date: Nov. 27, 2003

(54) Novel T-cell protein (TZON7), peptides and antibodies derived therefrom and uses thereof

(76) Inventor: Utku Nalan, Berlin (DE)

Correspondence Address: FROMMER LAWRENCE & HAUG, 745 FIFTH AVENUE- 10TH FL., NEW YORK, NY, 10151, US

(21) Appl. No.: 10/334,844

(22) Filed: Dec. 31, 2002

Related U.S. Application Data
(63) Continuation-in-part of application No. PCT/EP01/07610, filed on Jul. 3, 2001.

(30) Foreign Application Priority Data
Jul. 3, 2000 (EP) 00 114 234.8

(51) Int. Cl. A61K 48/00; C12Q 1/68; C07H 21/04; C12P 21/02; C07K 14/705; C12N 5/08

(52) U.S. Cl. 424/93.21; 435/6; 435/69.1; 435/320.1; 435/372; 530/350; 536/23.5

(57) ABSTRACT

Described are polynucleotide encoding TZON7 protein or a biologically fragment thereof. Furthermore, vectors are described comprising said polynucleotides and/or host cells transformed therewith. Additionally, antisense constructs to said polynucleotides are described. Furthermore, methods and uses for modulating immune responses through the TZON7 protein as well as pharmaceutical compositions comprising agents which act on the TZON7 protein are described. Also, the use of said polynucleotides, vectors, proteins or antibodies for the preparation of diagnostic and pharmaceutical compositions for use, inter alia, in organ transplantation, for the treatment of autoimmune, allergic or infectious diseases, or for treatment of tumors is provided.

### CROSS-REFERENCE TO RELATED APPLICATION

This application is a continuation-in-part of international application PCT/EP01/07610, filed Jul. 3, 2001, designating the U.S., published as WO 02/02619 on Jan. 10, 2002 and claims priority to European application number 00114234.8 filed Jul. 3, 2000.

Each of the foregoing applications and patents, each foregoing publication, and each document cited or referenced in each of the foregoing applications and patents, including during the prosecution of each of the foregoing applications and patents (“application and article cited documents”), and any manufacturer's instructions or catalogues for any products cited or mentioned in each of the foregoing applications and patents and articles and in any of the application and article cited documents, are hereby incorporated herein by reference. Furthermore, all documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art.

### FIELD OF THE INVENTION

The present invention relates to a polynucleotide encoding a TZON7 polypeptide comprising an amino acid sequence as disclosed herein, the expression of which is upregulated during the early stages of leukocyte/lymphocyte activation in response to alloantigens, or a biologically active fragment thereof. Furthermore, the present invention relates to a nucleic acid molecule of at least 15 nucleotides in length hybridizing specifically with a polynucleotide described herein or with a complementary strand thereof. In addition, the present invention pertains to vectors comprising polynucleotides encoding said TZON7 protein/polypeptide, (host) cells which comprise said polynucleotide(s) or said vectors, a TZON7 protein/polypeptide or biologically active fragments thereof, an antibody which specifically recognizes a TZON7 protein or a fragment thereof, or an antisense construct capable of inhibiting the expression of a polynucleotide encoding a TZON7 protein. Additionally this invention provides for diagnostic compositions and for methods of diagnosing biological conditions. Also, the invention relates to methods for identifying binding partners to a TZON7 protein and to methods for identifying leukocyte activating or co-stimulating compound or for identifying inhibitors of leukocyte activation and stimulation. Finally, the present invention relates to the use of the before described polynucleotide(s), vector(s), protein(s), antisense construct(s) for the preparation of compositions for diagnosing or the treatment of acute and chronic diseases, involving T-cell activation and Th1, and Th2 immune response, for the treatment of acute and chronic rejection of allo- and xeno-organ transplants and bone marrow transplantation, for the treatment of rheumatoid arthritis, lupus erythematodes, multiple sclerosis, encephalitis, vasculitis, diabetes mellitus, pancreatitis, gastritis, thyrolditis, for the treatment of (maligne) disorders of T, B or NK cells, for the treatment of asthma, lepramatosis, Helicobacter pylori associated gastritis or for the treatment of skin tumors, adrenal tumors or lung tumors, wound healing, growth disorders, inflammatory and/or infectious diseases.

Several documents are cited throughout the text of this specification. Each of the documents cited herein (including any manufacturer's specifications, instructions, etc.) are hereby incorporated herein by reference.

### BACKGROUND OF THE INVENTION

Immune activation is accompanied with sequential changes in the expression of various genes over several days and involves multiple signaling pathways [1]. Stimulation of T-cells is initiated by the interaction of antigen-specific T-cell receptors (TCR) with MHC bound antigenic peptides present on the surface of antigen presenting cells (APC), but full proliferative T-cell response requires additional costimulatory signals which are provided by the interaction of proteins expressed on the surface of T-cells and APC [2, 3, 4, 5]. In addition, a number of cytokines as well as other proteins are known to augment immune activation, although many of them appear not to be essential for the basic proliferative T-cell response [3, 6]. Moreover, a growing body of evidence indicates that the microtubule cytoskeleton of lymphocytes plays a major role in T-cell activation. Stimulation of T-cells was demonstrated to result in molecular rearrangement in the actin cytoskeleton leading to re-localization and concentration of signaling molecules in restricted areas of the cell membrane close to the bound APC [7, 8, 9].

Although considerable information on T-cell activation has been gathered in recent years, the complex molecular mechanisms of stimulation and signaling pathways are not completely understood. Since T-cell activation provides the central event in various types of inflammation as well as in autoimmune diseases and graft rejection, knowledge about the distinct steps and molecules involved in the stimulation process is of considerable biomedical importance, as they might provide targets for therapeutic modulation of the immune response. Therapeutic prevention of T-cell activation in organ transplantation and autoimmune diseases presently relies on panimmunosuppressive drugs interfering with downstream intracellular events.

Alloreactive CD4 or CD8 cells or specific alloantibodies are capable of mediating, inter alia, allograft rejection. The following immune mechanisms cause graft rejection by different mechanisms:

(a) alloactive T-cells can recruit and activate macrophages, initiating graft injury by “delayed-type” hypersensitivity response;

(b) alloactive cytotoxic T-cells are capable of directly lysing graft endothelial and parenchymal cells; and

(c) alloantibodies bind to endothelium, activate the complement system, and injure thereby graft blood vessels.

The various forms of (allograft) rejection imply a temporal sequence of events including hyperacute, acute vascular and acute cellular as well as chronic rejection.

Hyperacute rejection plays an important role in xenotransplantation and is due to natural antibodies (ref: Milford, E., Utku, A, N. Guidelines for use of immunogenetic tests organ transplantation, Manual of Clinical Laboratory Immunology, ASM Press 1997). Hyperacute rejection is mediated by preexisting antibodies that bind to endothelium and activate complement and is characterized by rapid thrombotic occlusion of the graft vasculature. In more recent clinical experience, hyperacute rejection of allografts is usually mediated by antibodies directed against protein alloantigens, such as foreign MHC molecules, or against less well described alloantigens expressed on vascular endothelial cells. Such antibodies generally arise as a result of prior exposure to alloantigens through blood transfusion, prior transplantation, or multiple pregnancies (loc. cit.). These antibodies are often of the IgG type. By testing recipients for the presence of such reactive antibodies with the cells potential donors, hyperacute rejection has been virtually eliminated from clinical allo-transplantation but remains a major problem in xenotransplantation.

Acute vascular rejection is mediated by IgG antibodies produced by B-cells against endothelial alloantigens and involves activation of complement. T-cells contribute to vascular injury by responding to alloantigens present on endothelial cells, leading to direct cell lysis of these cells, or the production of cytokines that recruit and activate inflammatory cells.

Acute cellular rejections is characterized by parenchymal cells and is usually accompanied by lymphocyte and macrophage infiltrates. These infiltrating leucocytes are responsible for the lysis of the graft. Several different effector mechanism may be involved in acute cellular rejection including CTL-mediated lysis, activated-macrophage-mediated lysis (as delayed type hypersensibility, DTH) and natural killer cells mediated lysis.

The identification of both antibody and lymphocytes as important effector mechanism in acute graft rejection suggests that this process is similar to normal antiviral immune responses. The basis of similarity probably arises from the fact that the foreign class I MHC molecules present in the graft are recognized as if they were self MHC molecules associated with endogenously synthesized foreign peptides.

Chronic rejection is characterized by fibrosis with loss of normal organ structures. The fibrosis may represent wound healing following the cellular necrosis of acute rejection or a form of DTH in which activated macrophages secrete mesenchymal cell growth factors, or alternatively chronic rejection is a response to chronic ischemia caused by injury of blood vessels. Vascular occlusion is due to proliferation of intimal smooth muscle cells, called accelerated or graft arteriosclerosis. This feature of chronic inflammation is characterized by fibrosis in autoimmune diseases such as lupus erythrematodes, sklerodermia and panarteritis nodosa. Chronic inflammatory immune response involves all parts of cellular and humoral immune system (T-, B-, NK-cells, monocytes) and is the response to the extensive production of autoantibodies and creation of immune complexes against different cellular components. This leads to multiple organ failure caused by significant tissues damage such as myositis, polyneuropathia, heart disease, vasculitis, etc.

Considering, inter alia, the above described temporal sequence events in allograft and xenograft rejection, it is desired to specifically modulate lymphocyte/leukocyte cell responses, i.e. to modulate T-, B-, NK-cells and/or monocyte responses during immunological processes. Furthermore, it is desired to specifically modulate immunological processes like, inter alia, autoimmunological events.

Specific modulation of the immune response remains, therefore, a longstanding goal in immunological research.

### SUMMARY OF THE INVENTION

The present invention relates to polynucleotides encoding an immune response modulating protein TZON7, Furthermore, the present invention relates to peptides and polypeptides derived therefrom as well as to antibodies. More particularly, the present invention relates to pharmaceutical compositions comprising said peptides and polypeptides as well as to pharmaceutical compositions comprising antibodies capable of inhibiting leukocyte stimulation through the immune response modulation protein TZON7. The present invention also relates to applications in the medical field that directly arise from the polynucleotides, protein, peptides, (poly)peptides, antisense constructs and antibodies described in this invention. Additionally, the present invention relates to a novel method for testing activators and inhibitors of leukocyte proliferation and/or lymphocyte activation, i.e. of leukocyte activation and/or stimulation. The pharmaceutical compositions, methods and uses of the invention are useful therapeutically and/or diagnostically in situations where it is desirable to modulate (antigen-specific) immune responses, e.g., inducing and maintain (antigen-specific) T-cell or B-cell non/unresponsiveness, wherein said non/unresponsiveness comprises the selective inhibition of immune cell subsets which are able to creating a response to specific antigen(s), inter alia, antigen(s) in transplanted tissue. The pharmaceutical compositions, methods and uses of the invention are furthermore useful to restore (antigen-specific) B-or T-cell responsiveness. For example, it may be necessary to induce or maintain “selective immune” unresponsiveness in a subject who has received an organ or bone marrow transplant to prevent graft rejection by inhibiting stimulation through the TZON7 protein in cells of the immune system such as T-cells, B-cells, NK-cells, monocytes and/or macrophages. In addition, T-cell unresponsiveness can be maintained by blocking TZON7 stimulation in a subject who has a autoimmune disease to alleviate symptoms of the autoimmune disease. In these cases, a TZON7 inhibitory agent is administered to the subject in an amount and over a period of time sufficient to maintain T-cell unresponsiveness. Alternatively, T-cell unresponsiveness can be reversed in a subject bearing a tumor to stimulate a tumor specific NK- and T-cell response or in a subject receiving a vaccine to enhance the efficacy of the vaccine. For example, it might be useful to induce or maintain the status of activation of the immune cells through vaccination with TZON7 peptides in a subject who developed a tumor to orchestrate the enhancement of immune response in T-, B-, NK-cells and/or monocytes.

### DETAILED DESCRIPTION OF THE PRESENT INVENTION

In view of the need of therapeutic means for the diagnosis and treatment of diseases related to immune responses of the human body, the technical problem of the invention is to provide means and methods for the modulation of immune cell responses which are particularly useful in organ transplantation and autoimmune diseases.

The solution to this technical problem is achieved by providing the embodiments characterized in the claims, namely a novel immune response modulating protein encoded by a cell immune response cDNA designated “TZON7” which comprises an amino acid sequence as depicted in SEQ ID NO: 2 and which exhibits a central role in leukocyte/lymphocyte activation and growth, wherein said leukocyte/lymphocyte activation refers to the activation of T-, B-, NK-cells and/or monocytes. TZON7 mRNA is transiently upregulated in the early phase of leukocyte/lymphocyte activation and in particular in T-cell activation.

In a first set of experiments, the TZON7 protein encoding cDNA has been cloned and characterized; see Example 1. Furthermore, the expression pattern of TZON7 was investigated after allo-stimulation of human leukocytes at time points 0 and 12 h and results obtained with alloactivated T-cells revealed an upregulation of TZON7 24 h after immune activation of the TZON7 gene. It is thus an excellent marker for diagnosis of the status of immune response in a subject. In addition, the full-length cDNA of TZON7 has been cloned, see Example 2.

The latter described results obtained in accordance with the present invention provide evidence for an essential role of TZON7 in the early events of leukocyte activation. Thus, targeting of TZON7 protein and its encoding gene provides a novel therapeutic approach for modulation of the immune response.

Accordingly, the invention relates to a polynucleotide encoding a TZON7 polypeptide, the expression of which is upregulated during the early stages of T-cell leukocyte/lymphocyte activation in response to alloantigens or a biologically active fragment thereof comprising a nucleic acid sequence selected from the group consisting of:

(I) DNA sequences encoding the amino acid sequence depicted in any one of SEQ ID NOS: 2, 4, or 9 to 13;

(II) the DNA sequence depicted in SEQ ID NO: 1 or 8;

(III) DNA sequences encoding a fragment or derivative of the protein encoded by the DNA sequence of (I) or (II);

(IV) DNA sequences the complementary strand of which hybridizes with and which is at least 70% identical to the polynucleotide as defined in any one of (I) to (III); and

(V) DNA sequences the nucleotide of which is degenerate to the nucleotide sequence of a DNA sequence of any one of (I) to (IV);

The term “TZON7 protein”, in accordance with the present invention, denotes a protein involved in the signal transduction of leukocyte/lymphocyte activation and/or proliferation and down-regulation which results in suppressing leukocyte/lymphocyte, preferably T-, B-, NK-cell and/or monocyte proliferation in response to alloactivation in a mixed lymphocyte culture when exogeneously added to the culture. In accordance with this invention it has been surprisingly found that a cDNA is differentially expressed in alloactivated lymphocytes, i.e. human T-cells. This differentially expressed cDNA was termed TZON7 and initially it was shown that this cDNA encodes for a protein sequence which is 18% homology to zonadhesin (GenBank accession number: U83191)

Here, it has surprisingly been found that said TZON7 protein/polypeptide plays an important role in the differentiation of quiescent T-cells to activate T-cells after alloantigen stimulation and/or cell activation/proliferation processes of B-cells, NK-cells and/or monocytes after stimulation by said allo/autoantigens or xenoantigens or by antigens from, inter alia, pathological agents, like viruses (viral agents), bacteria, etc.

The term “TZON7” denotes proteins/polypeptides, in accordance with this invention, which are identical to the TZON7 protein/polypeptide as described herein (see SEQ ID NO: 2 or 9 and FIG. 2) and the term comprises, furthermore, functional homologues of said protein/polypeptide such as peptides comprising an amino acid sequence as depicted in any one of SEQ ID NOS: 4 or 10 to 13. It can be concluded that TZON7 molecule is directly involved in the initiating of the immune response and might be an important target molecule for modulating the immune response.

The term “leukocyte/lymphocyte” generally denotes all kinds of white blood cells and preferably refers to monocytes and lymphocytes (B-T and NK cells), either in combination or individually. Thus, it should be understood that the term leukocyte may also be used herein so as to refer to individual species of leukocytes such as T-cells only.

In accordance with this invention the term “TZON7 polypeptide comprising an amino acid sequence as depicted in SEQ ID NO: 2 or 9” denotes a polypeptide comprising exactly said specific sequence but also comprises polypeptides which comprise a sequence which shows at least homology to said amino acid sequence as depicted in SEQ ID NO: 2. It is understood that said amino acid molecule may also comprise alternatives, deletions, duplications, additions, substitutions and/or silent mutations. Such silent mutations may, inter alia, comprise conservative amino acid replacements. The same applies to the immunologically and/or biologically active peptides of the present invention.

The above described polynucleotide/nucleic acid molecule encoding a TZON7 polypeptide as defined herein above also comprise polynucleotides/nucleic acid molecules which encode “variants” of said TZON7 polypeptide. Such “variants” refer to polynucleotides (and/or (poly)peptides) differing from the polynucleotides and/or (poly)peptides of the invention, but retain essential properties thereof, as, inter alia, is upregulation during early stages of leukocyte/lymphocyte activation in response to xenoantigens. It is preferred that said variants are overall (closely) similar, and, preferably, in some regions identical to the polynucleotides and (poly)peptides described herein. The term “variant” in accordance with this invention comprises, but is not limited to allelic variants, synthetically produced variants or genetically engineered variants.

The term “biologically active fragment thereof” refers to peptides and polypeptides that are derived from said TZON7 protein and that are capable of effecting the same or similar activity or at least one of said activities of TZON7 (see SEQ ID NO: 2 or 9, and SEQ ID NOS: 4, 10 to 13).

In accordance with the present invention, a gene induced in the early stage of T-cell activation has been identified by examining mRNA expression in alloactivated human lymphocytes. Differential display-reverse transcription PCR analysis revealed a 135 bp cDNA fragment which was upregulated 24 h after allostimulation of a human T-cell line; see Example 1. The corresponding (complete) cDNA named TZON7 (comprising 135 bp/predicted 44 amino acids). The deduced stretch protein shares 18% homology with human zonadhesin molecule. TZON7 is expected to function in cell proliferation and differentiation events during T-cell and/or general leukocyte activation.

Furthermore, the DNA sequence depicted in SEQ ID NO. 1 (the 135 base pair fragment AB6+m13f described in Example 1) was used to identify and clone a corresponding full-length cDNA encoding TZON7 polypeptide from cDNA libraries by conventional means and methods; see Example 2. A full length cDNA was obtained and sequence analysis revealed an 822 base pair cDNA (SEQ ID NO. 8) predicting a protein length of 274 amino acids (SEQ ID NO. 9) and thus a protein of a predicted molecular weight of about 29428.02 Dalton. Accordingly, in a preferred embodiment the polynucleotide of the invention encodes TZON7 polypeptide of about 29.4±1 kDa in its non-glycosylated form. Furthermore, its estimated pl is about 9.49. Its amino acid composition is given in Example 2.

The translated aminoacid sequence of 274 amino acids does share very high hologies (>80% identities) with murine proteins AK015954 and AK015299. Beyond this, homologies has been found to be present in “Mitochondrial Carrier Protein Family, Pet8p′ in S. cerevisiae (43% identities).

The function of the murine proteins is so far unknown. The proteins of the Pet8p family play an essential role in cellular energy metabolism. Given the sequence homology between TZON7 and “Mitochondrial Carrier Protein family Pet8p”, it is likely that TZON7 could be important for growth of activated T cells and possible applied as a potential therapeutic marker or may provide novel approaches for monitoring human organ transplantation and leukemia. Means and methods to test the biological activity of the (poly)peptides of the invention, antibodies, antisense constructs and other compounds described in the general description of the invention can be performed as described in the examples of, for example, WO99/11782 and WO01/32614 which herewith are incorporated by reference.

Furthermore, antigenicity analysis revealed at least four regions with highest immunogenicity; see FIG. 4 and SEQ ID NOs 10 to 13. Peptides comprising anyone of these immunologically active amino acid sequences are expected to be particular suitable for, e.g., raising antibodies and/or induction of immunological responses and T-cell modulation. Accordingly, in another preferred embodiment the polynucleotide of the present invention encodes a TZON7 (poly)peptide comprising one or more of the amino acid sequences depicted in SEQ ID NOs 10 to 13. Preferably, said (poly)peptides are about 15 to 100 amino acids in length, more preferably 15 to 50, and most preferably 15 to 40 amino acids. Likewise, the present invention relates to polynucleotides encoding a polypeptide that is recognized by polyclonal and/or monoclonal antibodies raised against a peptide comprising anyone of the above-identified immunogenic regions. For example, the polynuleotide or polypeptide of the present invention can be identified by first raising antibodies against the peptide comprising, e.g., the amino acid sequence depicted in SEQ ID NO. 10 according to conventional methods and then testing a protein in question or a sample comprising such protein on a dot or Western blot with said antibodies. In case, a signal is determined due to the binding of the antibody to the protein in question, the polypeptide, or optionally its corresponding encoding DNA can be identified. Furthermore, preferably monoclonal antibodies raised against the mentioned peptides can be used to identify and isolate TZON7 proteins or fragments thereof by, for example, immunochromatography. All these methods are well within the skill of the person skilled in the art.

From the above it is evident that the nucleotide sequences depicted in SEQ ID NOS: 1 and 8 encode a novel immune response modulating protein. By the provision of these nucleotide sequences it is now possible to isolate identical or similar polynucleotides which code for proteins with the biological, immunological activity of TZON7 from other species or organisms. Said nucleotide sequences may be employed, in accordance with this invention, in the pharmaceutical compositions, uses and/or methods described herein. Well-established approaches for the identification and isolation of such related sequences are, for example, the isolation from genomic or cDNA libraries using the complete or part of the disclosed sequence as a probe or the amplification of corresponding polynucleotides by polymerase chain reaction using specific primers.

Thus, the invention also relates to polynucleotides which hybridize to the above described polynucleotides and differ at one or more positions in comparison to these as long as they encode a TZON7 protein as defined above. Such molecules comprise those which are changed, for example, by deletion(s), insertion(s), alteration(s) or any other modification known in the art in comparison to the above described polynucleotides either alone or in combination. Methods for introducing such modifications in the polynucleotides of the invention are well-known to the person skilled in the art; see, e.g., Sambrook et al. (Molecular cloning; A Laboratory Manual, Second Edition, Cold Spring Harbor Laboratory Press; Cold Spring Harbor N.Y. (1989)). The invention also relates to polynucleotides the nucleotide Sequence of which differs from the nucleotide sequence of any of the above-described polynucleotides due to the degeneracy of the genetic code.

With respect to the DNA sequences characterized under (IV) above, the term “hybridizing” in this context is understood as referring to conventional hybridization conditions, preferably such as hybridization in 50%formamide/6×SSC/0.1%SDS/100 μg/ml ssDNA, in which temperatures for hybridization are above 37° C. and temperatures for washing in 0.1×SSC/0.1%SDS are above 55° C. Most preferably, the term “hybridizing” refers to stringent hybridization conditions, for example such as described in Sambrook, supra.

Inventive nucleic acid molecules include nucleic acid molecules having at least 70% identity or homology or similarity with the above mentioned polynucleotides or probes or primers derived therefrom such as at least 75% identity or homology or similarity, preferably at least 80% identity or homology or similarity, more preferably at least 85% identity or homology or similarity such as at least 90% identity or homology or similarity, more preferably at least 95% identity or homology or similarity such as at least 97% identity or homology or similarity. The nucleotide sequence similarity or homology or identity can be determined using the “Align” program of Myers and Miller, (“Optimal Alignments in Linear Space”, CABIOS 4, 11-17, 1988) and available at NCBI.

Alternatively or additionally, the terms “similarity” or “identity” or “homology”, for instance, with respect to a nucleotide sequence, is intended to indicate a quantitative measure of homology between two sequences. The percent sequence similarity can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence similarity of 75% with the sequence AATCAATC (N_{ref}=8; N_{dif}=2).

Alternatively or additionally, “similarity” with respect to sequences refers to the number of positions with identical nucleotides divided by the number of nucleotides in the shorter of the two sequences wherein alignment of the two sequences can be determined in accordance with the Wilbur and Lipman algorithm (Wilbur and Lipman, 1983 PNAS USA 80:726), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity with DNA sequences, thymidine (T) in the DNA sequence is considered equal to uracil (U) in the RNA sequence.

Particularly preferred are polynucleotides which share 70%, preferably at least 85%, more preferably 90-95%, and most preferably 96-99% sequence identity with one of the above-mentioned polynucleotides and have the same biological activity. Such polynucleotides also comprise those which are altered, for example by nucleotide deletion(s), insertion(s), substitution(s), addition(s), and/or recombination(s) and/or modification(s) known in the art either alone or in combination in comparison to the above-described polynucleotides. Methods for introducing such modifications in the nucleotide sequence of the polynucleotide of the invention are well known to the person skilled in the art. Thus, the pharmaceutical composition(s), use(s) and method(s) of the present invention may comprise any polynucleotide that can be derived from the above described polynucleotides by way of genetic engineering and that encode upon expression a TZON7 protein or a biologically active fragment thereof.

It is also immediately evident to the person skilled in the art that regulatory sequences may be added to the polynucleotide as defined herein and employed in the pharmaceutical composition, uses and/or methods of the invention. For example, promoters, transcriptional enhancers and/or sequences which allow for induced expression of the polynucleotide of the invention may be employed. A suitable inducible system is for example tetracycline-regulated gene expression as described, e.g., by Gossen and Bujard (Proc. Natl. Acad. Sci. USA 89 (1992), 5547-5551) and Gossen et al. (Trends Biotech. 12 (1994), 58-62).

In a preferred embodiment the polynucleotide of the invention encodes a polypeptide comprising an amino acid sequence as depicted in SEQ ID NO:2 or 9 or a biologically active fragment thereof.

In a further embodiment, the invention relates to a nucleic acid molecules of at least 15 nucleotides in length hybridizing with a polynucleotide as described above or with a complementary strand thereof. Specific hybridization occurs preferably under stringent conditions and implies no or very little cross-hybridization with nucleotide sequences encoding no or substantially different Proteins. Such nucleic acid molecules may be used as probes and/or for the control of gene expression. Nucleic acid probe technology is well known to those skilled in the art who will readily appreciate that such probes may vary in length. Preferred are nucleic acid probes of 17 to 35 nucleotides in length. Of course, it may also be appropriate to use nucleic acids of up to 100 and more nucleotides in length. Said nucleic acid probes are particularly useful for various pharmaceutical and/or diagnostic applications. On the one hand, they may be used as PCR primers for amplification of polynucleotides encoding TZON7 proteins and/or is homologues and may, thereby, serve as useful diagnostic tools. Another application is the use as a hybridization probe to identify polynucleotides hybridizing to the polynucleotides encoding TZON7 by homology screening of genomic DNA libraries. Nucleic acid molecules employed in this preferred embodiment of the invention which are complementary to a polynucleotide as described above may also be used for repression of expression of a gene comprising such a polynucleotide, for example due to an antisense or triple helix effect or for the construction of appropriate ribozymes (see, e.g., EP-A1 0 291 533, EP A1 0 321 201, EP-A2 0 360 257) which specifically cleave the (pre)-mRNA of a gene comprising a polynucleotide as described herein above. Selection of appropriate target sites and corresponding ribozymes can be done as described for example in Steinicke, Ribozymes, Methods in Cell Biology 50, Galbraith et al. eds Academic Press, Inc. (1995), 449-460. Standard methods relating to antisense technology have also been described (Melani, Cancer Res. (1991), 2897-2901). Said antisense or triple helix effect as well as the construction of relevant ribozymes is/are partially useful in pharmaceutical compositions to be employed for the suppression of the immune system, e.g., in autoimmune diseases, for the treatment of rejection events during or after transplantation, etc. Furthermore, the person skilled in the art is well aware that it is also possible to label such a nucleic acid probe with an appropriate marker for specific (Inter alia, diagnostic) applications, such as for the detection of the presence of a polynucleotide as described herein above in a sample derived from an organism.

The above described nucleic acid molecules may either be DNA or RNA or a hybrid thereof. Furthermore, said nucleic acid molecule may either contain, for example, thioester bonds and/or nucleotide analogues, commonly used in oligonucleotide antisense approaches. Said modifications may be useful for the stabilization of the nucleic acid molecule against endo- and/or exonucleases in the cell. Said nucleic acid molecules may be transcribed by an appropriate vector containing a chimeric gene which allows for the transcription of said nucleic acid molecule in the cell. Such nucleic acid molecules may further contain ribozyme sequences as described above.

In this respect, it is also to be understood that the polynocleotide to be used in the invention can be employed for “gene targeting” and/or “gene replacement”, for restoring a mutant gene or for creating a mutant gene via homologous recombination; see for example Mouellic, Proc. Nati. Acad. Sci. USA, 87 (1990), 4712-4716; Joyner, Gene Targeting, A Practical Approach, Oxford University Press.

In a particular preferred embodiment of the present invention, the polynucleotides as defined herein above may be employed in vaccination approaches. Such vaccination approaches may be, inter alia, useful in prevention or treatment of malignant diseases, for example in the prevention or therapy of tumors of the hematopoietic system. Vaccination approaches employing nucleic acid molecules are well known in the art and are described, inter alia, in Leither (2000), Vaccine 18, 765-777.

In a preferred embodiment said nucleic acid molecules are labeled. Said labels may comprise radiolabels or fluorescence labels. In another preferred embodiment said nucleic acid molecules may be used for the suppression of TZON7 expression. Particularly preferred in this embodiment are the above described hybridizing nucleic acid molecules.

The polynucleotide as employed in accordance with this invention and encoding the above described TZON7 protein or (a) biologically active fragment(s) thereof may be, e.g., DNA, cDNA, RNA or synthetically produced DNA or RNA or a recombinantly produced chimeric nucleic acid molecule comprising any of those polynucleotides either alone or in combination. Preferably said polynucleotide is part of a vector. Such vectors may comprise further genes such as marker genes which allow for the selection of said vector in a suitable host cell and under suitable conditions. Preferably, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in prokaryotic host cells comprise, e.g., the P_{L}, lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40.enhancer or a globin intron in mammalian and other animal cells. Beside elements which are responsible for the initiation of transcription such regulatory elements may also comprise transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (In-vitrogene), or pSPORT1 (GIBCO BRL).

Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the protein of the invention may follow; see, e.g., the appended examples. In one preferred embodiment of the present invention antisense constructs are made based on the polynucleotide encoding TZON7 (or (a) biologically active fragment(s) thereof) and combined with an appropriate expression control sequence.

The present invention also relates to vectors which comprise a polynucleotide useful in the present invention, host cells which are genetically engineered with vectors of the invention and the production of polypeptides useful in the present invention by such techniques.

For recombinant production, host cells can be genetically engineered to incorporate expression systems or polynucleotides of the invention. Introduction of a polynucleotide into the host cell can be effected by methods described in many standard laboratory manuals, such as Davis et al Basic Methods in Molecular Biology (1986) and Sambrook et al, Molecular Cloning A Laboratory Manual, Cold Spring Harbor Laboratory (1989) N.Y., such as calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as streptococci, staphylococci, E. Coli, streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al. Methods which are well known to those skilled in the art can be used to construct recombinant vectors; see, for example, the techniques described in Sambrook, supra, and Ausubel, Current Protocols in Molecular Biology, Green Publishing Associates and Wiley Interscience, N.Y. (1989).

Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

In the present invention the polynucleotide may be delivered to a target cell population, either ex vivo or in vivo, by any suitable Gene Delivery Vehicle.

This includes but is not restricted to, DNA, formulated in lipid or protein complexes or administered as naked DNA via injection or biolistic delivery, viruses such as retroviruses, adenoviruses, herpes viruses, vaccinia viruses, adeno associated viruses. The GDV can be designed by a person ordinarily skilled in the art of recombinant DNA technology and gene expression to express the fusion protein at appropriate levels and with the cellular specificity demanded by a particular application.

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a target cell. Optionally, once within the target cell, the vector may then serve to maintain the heterologous DNA within the cell or may act as a unit of DNA replication. Examples of vectors used in recombinant DNA techniques include plasmids, chromosomes, artificial chromosomes or viruses.

The vector can be delivered by viral or non-viral techniques.

Non-viral delivery systems include but are not limited to DNA transfection methods. Here, transfection includes a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), multivalent cations such as spermine, cationic lipids or polylysine, 1, 2,-bis (oleoyloxy)-3-(trimethylammonio) propane (DOTAP)-cholesterol complexes (Wolff and Trubetskoy 1998 Nature Biotechnology 16: 421) and combinations thereof.

Viral delivery systems include but are not limited to adenovirus vector, an adeno-associated viral (AAV) vector, a herpes viral vector, a retroviral vector, a lentiviral vector or a baculoviral vector.

Examples of retroviruses include but are not limited to: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV).

A detailed list of retroviruses may be found in Coffin et al (“Retroviruses” 1997 Cold Spring Harbour Laboratory Press Eds: JM Coffin, SM Hughes, HE Varmus pp 758-763).

Adenoviruses and adeno-associated viruses which have good specificity for epithelial cells are particularly preferred.

Other examples of vectors include ex vivo delivery systems, which include but are not limited to DNA transfection methods such as electroporation, DNA biolistics, lipid-mediated transfection, compacted DNA-mediated transfection.

Thus, nucleic acid vectors according to the invention may be capable of delivery preferentially to the target cell. For example in the case of a retroviral vector, the retroviral envelope protein may be capable of directing the vector to a particular cell type or cell types. For that purpose, the envelope protein may be a modified envelope protein adapted to have a specific targeting ability, or it may be a selected envelope protein derived from a different viral or retroviral source and having the desired targeting ability.

Preferably, the nucleic acid in a vector according to the invention is operatively linked to an expression control sequence capable of causing preferential expression of the fusion protein in the target cell. The expression control sequence may be for example a promotor or enhancer which is preferentially active in certain cell types including the target cell, or a promotor or enhancer which is preferentially active under certain conditions.

The term “promoter” is used in the normal sense of the art, e.g. an RNA polymerase binding site in the Jacob-Monod theory of gene expression.

The term “enhancer” includes a DNA sequence which binds to other protein components of the transcription initiation complex and thus facilitates the initiation of transcription directed by its associated promoter.

In accordance with the above, the present invention relates to (a) vector(s), particularly (a) plasmid(s), cosmid(s), virus(es) and bacteriophage(s) used conventionally in genetic engineering that comprise a polynucleotide encoding a TZON7 protein and/or (a) functional fragment(s) thereof (as defined herein above). Alternatively, the polypeptides and vectors to be employed in accordance with this invention can be reconstituted into liposomes for delivery to target-cells of the immune system. The here described vectors containing the polynucleotides described herein above can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

In a more preferred embodiment, the present invention provides for a vector as defined herein above, wherein said polynucleotide or nucleic acid molecule is operably linked to regulatory sequences allowing for the transcription and, optionally, expression of said acid molecules.

In a still further embodiment, the present invention relates to a cell, preferably a host cell, comprising the polynucleotide or vector described above. Preferably, said cell is a eukaryotic, most preferably a mammalian cell if therapeutic uses of the protein are envisaged. Of course, yeast and less preferred prokaryotic, e.g., bacterial cells may serve as well, in particular if the produced protein is used as a diagnostic means or if said protein is employed in methods as described herein above.

The polynucleotide or vector described herein which is present in the host cell may either be integrated into the genome of the host cell or it may be maintained extrachromosomally.

The term “prokaryotic” is mean to include all bacteria which can be transformed or transfected with a DNA or RNA molecules for the expression of a protein of the invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells. Depending upon the host employed in a recombinant production procedure, the protein encoded by the polynucleotide of the present invention may be glycosylated or may be non-glycosilated. TZON7 proteins as employed in accordance with the present invention may also include an initial menthionine amino acid residue. A polynucleotide as described herein can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, supra). The genetic constructs and methods described therein can be utilized for expression of the TZON7 protein in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Furthermore, transgenic animals, preferably mammals, comprising nucleic acid molecules/polynucleotides as defined herein may be used for the large scale production of the TZON7 protein and/or for the large scale production of pharmaceutical compositions described herein.

Alternatively, an animal, preferably mammalian cell naturally having a polynucleotide described herein present in its genome can be used and modified such that said cell expresses the endogenous gene corresponding to the polynucleotide described herein above under the control of an heterologous promoter. The introduction of the heterologous promoter which does not naturally control the expression of the polynucleotide of the invention can be done according to standard methods, see supra. Suitable promoter include those mentioned hereinbefore.

In this context, it should be mentioned that a method for the production of a TZON7 protein or a biologically active fragment thereof may comprise:

(a) culturing a host described herein above under conditions allowing for the expression of the protein; or

(b) in vitro translation of the polynucleotide encoding TZON7 and/or a biologically active fragment thereof;

and recovering the protein (or a fragment thereof) produced in (a) or (b).

The transformed hosts can be grown in fermentors and cultured according to techniques known in the art to achieve optimal cell growth. The TZON7 protein and/or biological active fragments thereof can then be isolated from the growth medium, cellular lysates, or cellular membrane fractions. Once expressed, the protein of the present invention can be purified according to standard procedures of the art, including ammonium sulfate precipitation, affinity columns, column chromatography, gel electrophoresis and the like; see, Scopes, “ProteinPurification”, Springer-Verlag, N.Y. (1982). Substantially pure proteins of at least about 90 to 95% homogeneity are preferred, and 98 to 99% or more homogeneity are most preferred, for pharmaceutical uses. Once purified, partially or to homogeneity as desired, the proteins may then be used therapeutically (including extracorporeally) or in developing and performing assay procedures.

Hence, in a further embodiment, the present invention relates to a TZON7 protein or a biological active fragment thereof encoded by polynucleotide described herein above or produced by a method of as above. It will be apparent to those skilled in the art that the TZON7 protein or a (biologically active) fragment thereof can be further coupled to other moieties as described above for, e.g., drug targeting and imaging applications, i.e. pharmaceutical and/or diagnostic uses. Such coupling may be conducted chemically after expression of the protein to site of attachment or the coupling product may be engineered into the protein of the invention at the DNA level. The DNAs are then expressed in a suitable host system, and the expressed proteins are collected and renatured. If necessary, TZON7 protein/polypeptides may by particularly useful in (a) pharmaceutical setting(s) where specific leukocyte activation should be controlled. As mentioned herein below, specific overexpression of TZON7 proteins or (biologically active) fragments thereof may be obtained by gene therapeutic approaches. As documented in the appended examples, TZON7 expression is induced by activation of cells of the immune system. Without being bound by theory, it is therefore envisaged that one function of TZON7 is the control of the cell activation events in the immune system.

Furthermore, the provision of the TZON7 protein as described herein above enables the production of TZON7 specific antibodies. In this respect, hybridoma technology enables production of cell lines secreting antibodies to essentially any desired substance that produces an immune response. RNA encoding the light and heavy chains of the immunoglobulin can then be obtained from the cytoplasm of the hybridoma. The 5′ end portion of the mRNA can be used to prepare cDNA to be inserted into an expression vector. The DNA encoding the antibody or is immunoglobulin chains can subsequently be expressed in cells, preferably mammalian cells.

Depending on the host cell, renaturation techniques may be required to attain proper conformation of the antibody. If necessary, point substitutions seeking to optimize binding may be made in the DNA using conventional cassette mutagenesis or other protein engineering methodology such as is disclosed herein.

Thus, the present invention also relates to an antibody specifically recognizing TZON7 protein or (a) fragment(s) (peptides, polypeptides) thereof.

In a preferred embodiment of the invention, said antibody comprised in said pharmaceutical composition is a monoclonal antibody, a single chain antibody, humanized antibody, or fragment thereof that specifically binds said peptide or polypeptide also including bispecific antibody, synthetic antibody, antibody fragment, such as Fab, Fv or scFv fragments etc., or a chemically modified derivate of any of these. Monoclonal antibodies can be prepared, for example, by the techniques as originally described in Kohler and Milstein, Nature 256 (1975), 495, and Galfré, Meth. Enzymol. 73 (1981), 3, which comprise the fusion of mouse myeloma cells to spleen cells derived from immunized mammals with modifications development by the art. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988. When derivates of said antibodies are obtained by the phage display technique, surface plasmon resonance as employed in the BIAcore system can be used to increase the efficiency of phage antibodies which bind to an epitope of the peptide or polypeptide of the invention (Schier, Human Antibodies Hybridomas 7 (1996), 97-105; Malmborg, J. Immunol. Methods 183 (1995), 7-13). The production of chimeric antibodies is described, for example, in WO89/09622. Methods for the production of humanized antibodies are described in, e.g., EP-A1 0 239 400 and WO90/07861. A further source of antibodies to be utilized in accordance with the present invention are so-called xenogenic antibodies. The general principle for the production of xenogenic antibodies such as human antibodies in mice is described in, e.g., WO91/10741, WO94/02602, WO96/34096 and WO96/33735. Antibodies to be employed in accordance with the invention or their corresponding immunoglobulin chain(s) can be further modified using conventional techniques known in the art, for example, by using amino acid deletion(s), insertion(s), substitution(s), addition(s), and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning A Laboratory Manual, Cold Spring Harbor Laboratory (1989) N.Y. It is particularly preferred that the here described

Pharmaceutical compositions comprise antibodies/antibody contructs which may be employed in intracellular settings. Such antibody constructs/antibodies are well known in the art and are, inter alia, described in Lener (2000), Eur. J. Biochem. 267, 1196-1205, who described intracellular antibodies against p21 ras.

In a still further embodiment, the present invention relates to a cell that has been modified to express a TZON7 protein or an antibody as described herein. This embodiment may be well suited for, e.g., restoring B and/or T-cell responsiveness to an antigen, in particular if the antibody of the invention capable of stimulating T-cell proliferation is expressed in a form suitable to be presented on the cell surface.

The invention also relates to an antisense construct capable of inhibiting the expression of the polynucleotide of the invention as described above or characterized in claim 1.

The invention furthermore relates to a pharmaceutical composition comprising the polynucleotides, nucleic acid molecules, vectors, cells, proteins, peptides, antibodies or antisense construct of the invention.

The invention also relates to (a) pharmaceutical composition(s) comprising an antisense construct capable of inhibiting the expression of a polynucleotide encoding TZON7 (and/or (a) biologically active fragment(s) thereof) as defined herein above. Such antisense constructs/oligonucleotides are particularly useful in the down regulation of leukocyte/lymphocyte responses/activations. Therefore, the here described pharmaceutical compositions comprising (specific) antisense constructs which are capable of inhibiting the expression of TZON7 may be particularly useful in the treatment and/or prevention of pathological or medical situations where an immunoactivation is not desired. These situations comprise, but are not limited to, treatment of acute and chronic rejections of allo- and xeno(organ)transplants or bone marrow transplantations, inflammation processes and/or allergies. The use of antisense oligonucleotides/constructs is well known in the art and described, inter alia, in Irizawa (1995), Clin. Exp. Immunology 100, 383-389 or Boeve (1994), J. leukocyte Biol. 55, 169-174.

In yet another embodiment the present invention relates to a pharmaceutical composition(s) described herein for use in cell or organ transplantation, for the treatment of autoimmune, allergic or infectious diseases, for the treatment of tumors or for the improvement of allograft or xenograft tolerance.

An example for the use of the Pharmaceutical composition of the Invention for improving allograft or xenograft tolerance is described with respect to administration of an LFA-3 and CD2 binding protein, respectively, in WO93/06852.

The pharmaceutical composition of the present invention may further comprise a pharmaceutically acceptable carrier. Examples of suitable pharmaceutical carriers are well known in the art and include phosphate buffered saline solutions, water, emulsions such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10⁶ to 10¹² copies of the DNA molecule. The composition of the invention may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsion. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsion or suspensions, including saline and buffered media. Parental vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. Furthermore, the pharmaceutical composition of the invention may comprise further agents such as T-cell, B-cell, NK-cell or monocyte costimulatory molecules or cytokines known in the art, or their inhibitors or activators depending on the intended use of the pharmaceutical composition.

Furthermore, it is envisaged by the present invention that the various polynucleotides and vectors encoding the above described peptides or polypeptides are administered either alone or in any combination using standard vectors and/or gene delivery systems, and optionally together with a pharmaceutically acceptable carrier or excipient. For example, the polynucleotide of the invention can be used alone or as part of a vector to express the (poly)peptide described herein in cells, for, e.g., gene therapy or diagnostics of diseases related to disorders of the immune system. The polynucleotides or vectors described herein are introduced into the cells which in turn produce the TZON7 protein (or (a) fragment(s) thereof). Subsequent to administration, said polynucleotides or vectors may be stably integrated into the gnome of the subject. On the other hand, viral vectors may be used which are specific for certain cells or tissues and persist in said cells. Suitable pharmaceutical carriers and excipients are well known in the art. The pharmaceutical compositions prepared according to the invention can be used for the prevention or treatment or delaying of different kinds of diseases, which are related to leukocyte, lymphocyte and/or monocyte related immunodeficiencies and malignancies such as multiple myeloma, T-, B-cell leukemia, infectious diseases related to T-, B-, NK-cell and monocyte proliferation, immune activation in refection of transplants, autoimmune disorders, allergy.

In another embodiment the present invention relates to a diagnostic composition comprising any one of the above described proteins, antibodies, (poly)peptides, polynucleotides, vectors or cells, and optionally suitable means for detection. The (poly)peptides and antibodies described above are, for example, suited for use in immunoassays in which they can be utilized in liquid phase or bound to a solid phase carrier. Examples of immunoassays in either a direct or indirect format. Examples of such immunoassays are the radioimmunoassay (RIA), the sandwich (immunometric assay) and the Western blot assay. The (poly)peptides and antibodies can be bound in many different carriers and used to isolate cells specifically bound to said polypeptides. Examples of well-known carriers include glass, polystyrene, polyvinyl chloride, polypropylene, polyethylene, polycarbonate. dextran, nylon, amyloses, natural and modified celluloses, polyacrylamides. agaroses, and magnetite. The nature of the carrier can be either soluble or insoluble for the purposes of the invention.

There are many different labels and method of labeling known to those of ordinary skill in the art. Examples of the types of labels which can be used in the present invention include enzymes, radioisotopes, colloidal metals, fluorescent compounds, chemiluminescent compounds, and bioluminescent compounds. The here described diagnostic compositions are particularly useful for the detection of an activated status of the immune system, in particular to detect activation of T-cells, B-cells, NK-cells and/or monocytes.

Said diagnostic compositions may also be use for methods for detecting expression of a polynucleotide encoding TZON7 (or its homologues) by detecting the presence of mRNa coding for a TZON7 protein which comprises obtaining mRNA from a cell and contacting the mRNA so obtained with a probe comprising a nucleic acid molecule of at least 15 nucleotides capable of specifically hybridizing with a polynucleotide encoding TZON7 (or its homologues) under suitable hybridizing conditions (see also supra), detecting the presence of mRNA hybridized to the probe, and thereby detecting the expression of the TZON7 protein (or its homologues) by the cell.

Furthermore, the invention comprises methods of detecting the presence of a TZON7 protein in a sample, for example, a cell sample, which comprises obtaining a cell sample from a subject, contacting said sample with one of the aforementioned antibodies under conditions permitting binding of the antibody to the TZON7 protein, and detecting the presence of the antibody so bound, for example, using immuno assay techniques such as radio-immunoassay or enzyme-immunoassay. Furthermore, one skilled in the art may specifically detect and distinguish polypeptides which are functional TZON7 proteins from a mutated forms which have lost or altered their leukocyte (T-cell, B-cell, etc.) stimulatory activity by using an antibody which either specifically recognizes a (poly)peptide which has TZON7 activity but does not recognize an inactive form thereof or which specifically recognizes an in inactive form but not the corresponding polypeptide having TZON7 activity. The antibodies as described in the present invention may also be used in affinity chromatography for purifying the TZON7 protein or above described (poly)peptides and isolating them from various sources. Said purified proteins/(poly)peptides may be employed in the pharmaceutical compositions, uses an/or method of the present invention.

In a further embodiment the invention relates to a method for diagnosing a pathological condition or a susceptibility to a pathological condition in a subject related to a disorder in the immune system comprising the steps of determining the presence or absence of a mutation in the polynucleotide if the indention and diagnosing a pathological condition or a susceptibility to a pathological condition based on the presence or absence of an mutation in the polynucleotide of the invention.

Furthermore, the indention relates to a method of diagnosing a pathological condition or susceptibility to a pathological condition in a subject related to a disorder in the immune system comprising the steps of determining the presence or amount of expression of the protein of the invention in a biological sample and diagnosing a pathological condition or a susceptibility to a pathological condition based on the presence or amount of expression of the protein.

The invention also encompasses a method for diagnosing in a subject a predisposition (susceptibility) to a disorder associated with the expression of a TZON7 allele which comprises isolating DNA from victims of the disorder associated with the under- or over-expression of a TZON7 protein; digesting the isolated DNA with at least one restriction enzyme; electrophoretically separating the resulting DNA fragments on a sizing gel; contacting the resulting gel with a nucleic acid probe as described above capable of specifically hybridizing to DNA encoding a TZON7 protein and labeled with a detectable marker; detecting labeled bands on the gel which have hybridized to the labeled probe to create a band pattern specific to the DNA of victims of the disorder associated with the expression of a TZON7 protein; preparing the subject's DNA according to the above-mentioned steps to produce detectable labeled bands on a gel; and comparing the band pattern specific to the DNA of victims of the disorder associated with the expression of a TZON7 protein and the subject's DNA to determine whether the patterns are the same or different and to diagnose thereby predisposition to the disorder if the patterns are the same. The detectable markers of the present invention may be labeled with commonly employed radioactive labels, such as, for example, ³²P or ³⁵S, although other labels such as biotin or mercury as well as those described above may be employed as well. Various methods well-known the person skilled in the art may be used to label the detectable markers. For example, DNA sequences and RNA sequences may be labeled with ³²P or ³⁵S using the random primer method. Once a suitable detectable marker has been obtained, various methods well-known to the person skilled in the art may be employed for contacting the detectable marker with the sample of interest. For example. DNA-DNA, RNA-RNA and DNA-RNA hybridizations may be performed using standard procedures. Various methods for the detection of nucleic acids are well-known in the art, e.g., Southern and northern blotting, PCR. primer extension and the like. Furthermore, the mRNA, cRNA, cDNA or genomic DNA obtained from the subject may be sequenced to identify mutations which may be characteristic fingerprints of TZON7 mutations in disorders associated with the expression of TZON7 or mutated versions thereof. The present invention further comprises methods, wherein such a fingerprint may be generated by RFLPs of DNA or RNA obtained from the subject, optionally the DNA or RNA may be amplified prior to analysis, the methods of which are well known in the art. RNA fingerprints may be performed by, for example, digesting an RNA sample obtained from the subject with a suitable RNA-Enzyme, for example RNase T₁, Rnase T₂ or the like or a ribozyme and, for example, electrophoretically separating and detecting the RNA fragments on PAGE as described above or in the appended examples.

In another embodiment, the present invention relates to a pharmaceutical composition comprising an agent which stimulates a leukocyte through the TZON7 protein as described herein, and optionally a pharmaceutically acceptable carrier. As is immediately evident to the person skilled in the art, the provision of the TZON7 as an immunomodulating molecule opens up the way of alternative approaches for leukocytes stimulation and treating corresponding diseases. The agent that stimulates the proliferation of leukocytes or lymphocytes through the TZON7 protein is expected to markedly enhance the proliferation of leukocytes or lymphocytes of, e.g., (activated) T-cells and thus is capable of augmenting the immune response. Examples for this type of “vaccine” is described, e.g., in WO91/11194 and in the literature, e.g., referred to above. The agents to be employed in accordance with the present invention usually specifically bind and/or interact to TZON7 protein in order to exert their effect. Such agents can be identified in accordance with a method of the invention described below. Such agents also comprise promoters which can be inserted in front of the coding region of the TZON7 protein encoding gene, e.g., via gene transfer and homologous recombination in the 5′ untranslated region of the gene, see also supra. Such promoter may be regulated and thus permit the controlled expression of the TZON7 protein in certain cells.

Therefore, in a further aspect the present invention relates to a method for identifying a binding partner to a TZON7 polypeptide comprising:

(a) contacting a TZON7 polypeptide (protein) of the invention with a compound to be screened; and

(b) determining whether the compound effects an activity of the polypeptide (protein).

TZON7 polypeptides may be used to screen for molecules that bind to TZON7 or for molecules to which TZON7 binds. The binding of TZON7 and the molecule may activate (agonist),increase, inhibit (antagonist), or decrease activity of the TZON7 or the molecule bound. Examples of such molecules include antibodies (including single-chain antibodies), oligonucleotides, proteins (e.g., receptors), or small molecules preferably, the molecule is closely related to the natural binding partner of TZON7, e.g., a fragment of the binding partner, or a natural substrate, a “ligand”, a structural or functional mimetic; see, e.g., Collgan, Current Protocols in Immunology 1(2) (1991); Chapter 5. Similarly, the molecule can be closely related to the natural binding partner(s) with which TZON7 interacts, or at least, a fragment of said binding and/or interaction partner capable of being bound by TZON7 (e.g., active site). In either case, the molecule can be rationally designed using known techniques; see also infra. (A) potential binding partner(s) of TZON7 is/are G-protein interacting molecule(s).

Preferably, the screening for these molecules involves producing appropriate cells which express TZON7, either as a secreted protein of as a protein in or on the cell membrane. Preferred cells include cells from mammals, yeast, Drosophila, or E.coli. Cells expressing TZON7 (or cell membrane(s) containing the expressed polypeptide) are then preferably contacted with a test compound potentially containing the molecule to observe binding, stimulation, or inhibition of activity of either TZON7 or the molecule. The assay may simply test binding of a candidate compound to TZON7, wherein binding is detected by a label, or in an assay involving competition with a labeled competitor. Further, the assay may test whether the candidate compound results in a signal generated by binding to TZON7. Alternatively, the assay can be carried out using cell-free preparations. polypeptide/molecule affixes to a solid support, chemical libraries, or natural product mixtures. The assay may also simply comprise the steps of mixing a candidate compound with a solution containing the TZON7/molecule activity or binding to a standard.

Preferably, an ELISA assay can measure TZON7 level or activity in a sample (e.g., biological sample) using a monoclonal or polyclonal antibody. The antibody can measure TZON7 level or activity by either binding, directly or indirectly, to TZON7 or by competing with TZON7 for a substrate.

All of these above assays can be used as diagnostic or prognostic markers. The molecules discovered using these assays can be used to treat disease or to bring about a particular result in a patient (e.g., increase of immune response) by activating or inhibiting the TZON7/molecule. Moreover, the assays can discover agents which may inhibit or enhance the production of TZON7 from suitably manipulated cells or tissues.

Therefore, the invention includes a method of identifying compounds which bind to TZON7 comprising the steps of:

(a) incubating a candidate binding compound with TZON7; and

(b) determining if binding has occurred:

Moreover, the invention includes a method of identifying agonists/antagonists comprising the steps of:

(a) incubating a candidate compound with TZON7;

(b) assaying a biological activity as described above, and

(c) determining if a biological activity of TZON7 has been altered.

As mentioned hereinbefore, the polynucleotides encoding TZON7 (or (a) fragment(s) thereof) and polypeptides representing TZON7 (or (a) fragment(s) thereof) provide a basis for the development of mimetic compounds that may be inhibitors or activators of TZON7 or their encoding genes. It will be appreciated that the present invention also provides cell based screening methods that allow a high-throughput-screening (HTS) of compounds that may be candidates for such inhibitors and activators.

Furthermore, the invention relates to a method for indentifying leukocyte/lymphocyte activation or co-stimulating compounds or for identifying inhibitors of leukocyte/lymphocyte activation and stimulation comprising

(a) culturing leukocytes, lymphocytes or monocytes in the presence of the TZON7 protein, (poly)peptide, antibody, cell and/or the antisense constrict described above and, optionally, in the presence of a component capable of providing a detectable signal in response to leukocyte proliferation/activation, with a compound to be screened under conditions permitting interaction of the compound with the TZON7 protein, (poly)peptide, antibody or cell(s), and

(b) detection the presence or absence of a signal generated from the interaction of the compound with the cells.

The term “compound” in the method of the invention includes a single substance or a plurality of substances which may or may not be identical.

Said compound(s) may be comprised in, for example, samples, e.g., cell extracts from, e.g., plants, animals or microorganisms, Furthermore, said compounds may be known in the art but hitherto not known to be capable of inhibiting proliferation of leukocytes or not known to be useful as an immune response costimulatory factor, respectively. The plurality of compounds may be, e.g., added to a simple in vitro, to the vulture medium or injected into the cell.

If the sample containing (a) compound(s) is identified in the method of the invention, then it is either possible to isolate the compound from the original sample identified as containing the compound, in question or one can further subdivide the original sample, for example, if it consists of a plurality of different compounds, so as to reduce the number of different substances per sample and repeat the method with the subdivisions of the original sample. It can then be determined whether said sample or compound displays the desired properties by methods known in the art such as described herein and in the appended examples. Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art (see, e.g., EP-A-0 403 506) or by using and modifying the methods as described in the appended examples. Furthermore, the person skilled in the art will readily recognize which further compounds and/or cells may be used in order to perform the methods of the invention, for example, B-cells, interleukins, or enzymes, if necessary, that, e.g., convert a certain compound into the precursor which in turn stimulates or suppresses lymphocytes or monocyte activation or that provide for (co)stimulatory signals. Such adaptation of the method of the invention is well within the skill of the person skilled in the art and can be performed without undue experimentation.

Compounds which can be used in accordance with the method of the present invention include peptides, proteins, nucleic acids including cDNA expression libraries, antibodies, small organic compounds, ligands, peptidomimetics, PNAs and the like, Said compounds can also be functional derivatives or analogues of known leukocyte, lymphocyte (B-, T- or NK-cell) or monocyte activators or inhibitors. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art or as described, for example, in the appended examples, Furthermore, peptidomimetics and/or computer aided design of appropriate activators or inhibitors of leukocytes, lymphocytes, monocytes (like T-cell, B-cell, NK-cell) activation can be used, for example, according to the methods described below. Appropriate computer programs can be used for the identification of interactive sites if a putative inhibitor and the TZON7 protein (or its biologically active fragment(s)) by computer assistant searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. The results obtained from the above-described computer analysis can be used in combination with the method of the invention for e.g., optimizing known leukocyte activators or inhibitors. Appropriate peptidomimetics can also be identified by the synthesis of peptidomimetic combinatorial libraries through successive chemical modification and testing the resulting compounds, e.g., according to the methods described herein and in the appended examples. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of inhibitors or activators of leucocyte stimulation can be used for the design of peptidomimetic inhibitors or activators of leukocyte activation to be tested in the method of the invention (Rose, Biochemistry 35 (1996), 12933-12944; Rutenber, Bioorg. Med. Chem. 4 (1996),1545-1558).

In summary, the present invention provides methods for identifying compounds which are capable of modulating immune responses. Accordingly compounds identified in accordance with the method of the present invention to be inhibitors and activators, respectively, of immune response are also within the scope of the present invention.

Compounds found to enhance leukocyte proliferation may be used in the treatment of cancer or infections and related diseases. In addition, it may also be possible to specifically inhibit viral diseases, thereby preventing viral infection or viral spread. Compound identified as suppressors of leukocyte proliferation can be used, e.g., for treating skin conditions (see, e.g., WO93/06866) or in allogenic or xenogenic cell or organ transplantation in order to avoid graft refection; see also supra.

The compounds identified or obtained according to the method of the present invention are thus expected to be very useful in diagnostic and in particular for therapeutic applications.

Hence, in a further embodiment the invention relates to a method for the production of a pharmaceutical composition comprising formulating and optionally synthesizing the compound identified in step (b) of the above described methods of the invention in a pharmaceutically acceptable form. Hence, the present invention generally relates to a method of making a therapeutic agent comprising synthesizing the proteins, (poly)peptides, polynucleotides, vectors, antibodies or compounds according to the invention in an amount sufficient to provide said agent in a therapeutically effective amount to the patient. Methods for synthesizing these agents are well known in the art and are described, e.g. above.

The therapeutically useful compounds identified according to the method of the invention may be administered to a patient by any appropriate method for the particular compound, e.g., orally, intravenously, parenterally, transdermally, transmucosally, or by surgery or implantation (e.g., with the compound being in the form of a solid or semisolid biologically compatible and resorbable matrix) at or near the site where the effect of the compound is desired. Therapeutic doses are determined to be appropriate by one skilled in the art, see also supra.

Such useful compounds can be for example transacting factors which bind to the TZON7 protein described herein. Identification of transacting factors can be carried out using standard methods in the art (see, e.g., Sambrook, supra and Ausubel, supra). To determine whether a protein binds to the TZON7 protein, standard native gel-shift analyses can be carried out. In order to identify a transacting factor which binds to the TZON7 protein, the polypeptides and peptides described in this invention can be used as an affinity reagent in standard protein purification methods, or as a probe for screening an expression library. Once the transacting factor is identified, modulation of its binding to the TZON7 protein as described herein can be pursued, beginning with, for example, screening for inhibitors against the binding of the transacting factor to the TZON7 protein. Activation or repression of TZON7 specific genes could the be achieved in subjects by applying the transacting factor (or its inhibitor) or the gene encoding it, e.g., in a vector described in the embodiments hereinbefore. In addition, if the active form of the transacting factor is a dimer, dominant-negative mutants of the transacting factor could be made in order to inhibit its activity. Furthermore, upon identification of the transacting factor, further components in the pathway leading to activation (e.g. signal transduction) or repression of a gene encoding the TZON7 protein described herein can then be identified. Modulation of the activities of the components can the be pursued, in order to develop additional drugs and methods for modulating the expression or activity of the TZON7 protein.

In yet another embodiment the invention relates to a method for determining the status of the immune system comprising analyzing the presence of the polynucleotide or the protein of the invention.

Beside the above described possibilities to use the polynucleotides according to the invention for gene therapy and their use to identify homologous molecules, the described polynucleotides may also be used for several other applications, for example, for the identification of nucleic acid molecules which encode proteins which interact with the TZON7 protein described above. This can be achieved by assays well known in the art, for example, as describe in Scofield (Science 274 (1996), 2063-2065) by use of the so-called yeast “two-hybrid system”. In this system the (poly)peptide encoded by the polynucleotides according to the invention or a smaller part thereof is linked to the DNA-binding domain of the GAL4 transcription factor. A yeast strain expressing this fusion protein and comprising a lacZ reporter gene driven by an appropriate promoter, which is recognized by the GAL4 transcription factor, is transformed with a library of cDNAs which will express animal, preferably mammal proteins or peptides thereof fused to an activation domain. Thus, if a peptide encoded by one of the cDNAs is able to interact with the fusion protein comprising a (poly)peptide of the invention, the complex is able to direct expression of the reporter gene. In this way the polynucleotide according to the invention and the encoded peptide can be used to identify peptides and proteins interacting with TZON7 proteins.

Other methods for identifying compounds which interact with the TZON7 protein according to the invention or nucleic acid molecules encoding such molecules are, for example, the in vitro screening with the phage displays system as well as filter binding assays or “real time” measuring of interaction using, for example, the BIAcore apparatus (Pharmacia); see references cited supra. Furthermore, the present invention relates to the use of the polynucleotide, the nucleic acid molecule, the vectors, peptides, polypeptides, antibodies and cells described herein as well as compounds identified in accordance with a method of the invention described herein above for the preparation of a composition for diagnosing and/or the treatment of acute and chronic diseases involving T-cell activation and associated with Th1 and Th2 immune response, for the treatment of acute and chronic rejection of allo- and xeno organ transplants ad bone marrow transplantation, for the treatment of rheumatoid arthritis, lupus erythrematodes, multiple sclerosis, encephalitis, vasculitis, diabetes mellitus, pancreatitis, gastritis, thyroiditis, for the treatment of disorders (inter alia malignant disorders) of T-, B- or NK-cells, for the treatment of asthma, lepramatosis, Helicobacter pylori associated gastritis or for the treatment of skin tumors, adrenal tumors or lung tumors, wound healing, growth disorders, inflammatory and/or infectious diseases. It is particularly preferred that the polynucleotide encoding TZON7 (or (a) fragment(s) thereof) or the antibody as defined herein above is employed for the detection of leukocyte/lymphocyte activation and/or for the treatment of diseases linked to leukocyte/lymphocytes activation.

The polynucleotides, vectors, cells, proteins, (poly)peptides, antibodies, inhibitors, activators, pharmaceutical and diagnosis compositions, uses described herein above and methods of the invention can be used for the treatment of all kinds of diseases hitherto unknown as being related to or dependent on the modulation of TZON7. The pharmaceutical compositions, methods and uses of the present invention may be desirably employed in humans, although animal treatment is also encompassed by the methods and uses described herein.

In a further embodiment the invention relates to the use of the polynucleotides, nucleic acid molecules or antibodies of the invention for the detection of leukocyte activation as described herein above.

In a preferred embodiment said leukocyte is a B-cell, T-cell, NK-cell and/or monocyte.

These and other embodiments are disclosed and encompassed by the description and Examples of the present invention. Further literature concerning any one of the antibodies, methods, uses and compounds to be employed in accordance with the present invention may be retrieved from public libraries and databases, using for example electronic devices. For example the public database “Medline” may be utilized which is available on the Internet, for example under http://www.ncbi.nim.nih.gov/PubMed/medline.html. The sequence database Genbank can be accessed at http://www.ncbi.nim.nih.gov/. Further databases and addresses, such as, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

### BRIEF DESCRIPTION OF DRAWINGS

This disclosure may best be understood in conjunction with the accompanying drawings, incorporated herein be references, which show:

FIG. 1a: Nucleotide sequence of TZON7

FIG. 1b: Amino acid sequence of TZON7 cDNA

FIG. 2: Translation of TZON7 cDNA sequence

FIG. 3: Antigenicity blot of TZON7 amino acid sequence

FIG. 4: Antigenicity blot of TZON7 amino acid sequence

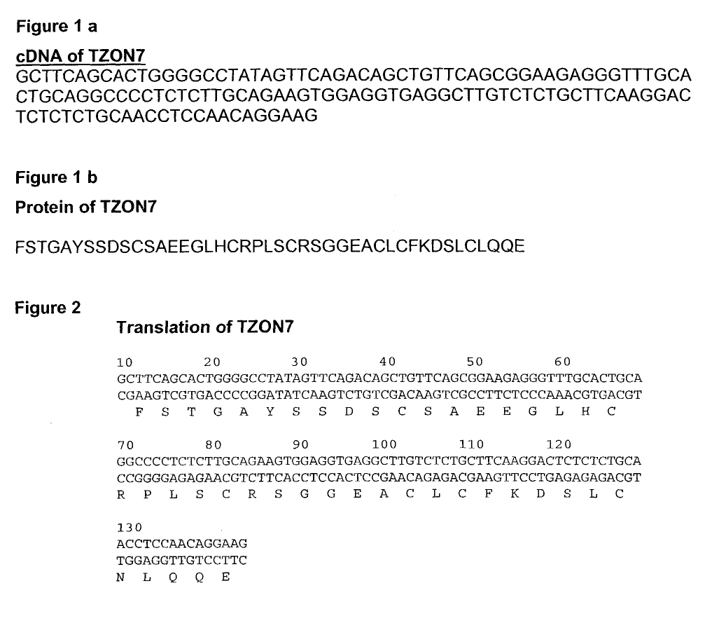

A better understanding of the present invention and of is many advantages will be had the following examples, given by way of illustration.

### EXAMPLE 1

### Identification of a Novel cDNA Fragment, TZON7, that is Differentially Expressed in Alloactivated Human T Cell Lines

To identify novel genes induced during the early stages of T cell activation in response to allo-antigens, differential display RT-PCR (DDRT-PCR) analysis of mRNA expression was performed at time 0 and 24 h after stimulation of a preconditioned human T cell line with allo-antigen. The preconditioned T cell line was prepared as follows: In conformance with institutional policies regarding human experimentation, peripheral blood lymphocytes (PBLs) were isolated from the healthy human volunteers using standard Ficoll centrifugation methods and diluted into RPMI containing 10% fetal calf serum. Isolated human PBLs (responder PBLs) were stimulated with equal numbers of irradiated (3000 rad, 13 min) stimulator PBLs from another healthy individual. Cells were co-cultured in tissue flasks at an initial concentration of 10⁶ cells/ml restimulated with stimulator cells three times in 10 day intervals prior to RNA isolation. Total RNA was isolated from cells at 0 and 24 h after last stimulation using the RNAzol B method (Tel-Test, Inc) and differential display was performed as described previously (Kojima et al., 1996). DDRT-PCR is a method which yields unbiased analysis of changes in message levels from cDNA amplified with multiple sets of primers followed by parallel 6% polyacrylamide gel electrophoresis. Briefly, 2 μg of total RNA was reverse transcribed using an oligo-dT primer and 200 U MMLV reverse transrciptase (Gibco/BRL). A 40 cycle PCR amplification with a total volume of 10 μl was performed by using 1 μg of cDNA, 1.25 mM MgCl₂, 50 mM KCl, 10 mM Tris-HCl (pH 8.3), 2.5 nM primer, 5 μCl ³⁵S-dATP, and 0.3 U Taq polymerase. The primers for the PCR amplification were: 5′-TGCTTCAGCACTGCC-3′ (SEQ ID NO:6) and −5′TTATTGTATTTGAAGTAA-3′ (SEQ ID NO:7). The PCR products were stored at 4° C. and separated by electrophoresis in 6% polyacrylamide-urea gels, transferred to filter paper, dried, and autoradiographed. The differentially expressed cDNA fragment was excised from the gel, eluted, reamplified, cloned into pBluescriptSK⁺ plasmid, and sequenced. Homology searches were performed using BLAST at NCBI. Alignments were performed using Geneworks 2.1.1.

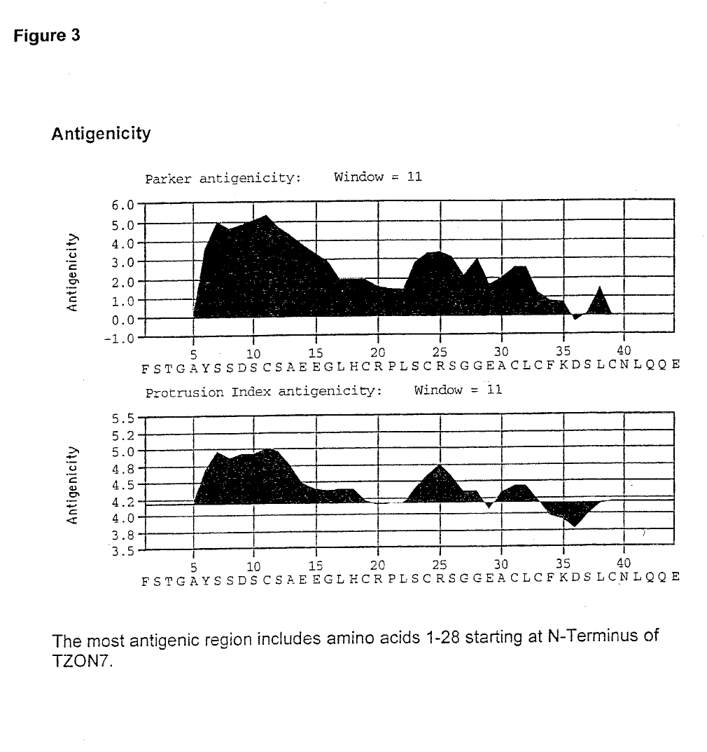

Analysis of the cDNAs showed several genes at 24 hours. One of the upregulated transcripts, TZON7, was reamplified, subcloned and 135 base pairs were sequenced. By searching GenBank, the deduced 44 amino acid sequence of TZON7 showed 18% identity to human zonadhesin molecule (GenBank accession number: U83191). Most antigenic region of the TZON7 molecule is localized between amino acid 5-14 and 22-33 as shown in FIG. 3.

### EXAMPLE 2

### Identification and Cloning of a TZON7 Full-Length cDNA

To obtain a full length cDNA, the 135 bp fragment obtained in Example 1 was used to screen cDNA libraries (OriGene, human peripheral blood leucocytes) and HPB-ALL (human acute lymphoblastic leukemia cell line) cDNA. The differentially expressed cDNA fragment was excised from the filter, eluted in 0.5 M ammonium acetate/1 mM EDTA, pH 8.3, and ethanol precipitated. The cDNA product was reamplified, electrophoresed in a 2% agarose gel and purified using Gene Clean kit (Quiagen). The recovered cDNA product was blunt-ended with Klenow enzyme (Gibco BRL) following standard protocols [11] and ligated into pBluescript SK⁺ vector. After labeling with α³²P[dCTP] (800 mCi/mmol, Amersham Inc.) using the random priming method [12], the cDNA fragment isolated from DDRT-PCR was used as a probe to screen the mentioned CDNA libraries. Hybridization was carried out for 24 h at 42° C. in 40% formamide, 10% Dextran sulfate, 4×SSC (1×SSC consists of 150 mM NaCl, 15 mM sodium citrate, pH 7.0), 0.8× Denhardt's solution (1× Denhardt's solution contains 0.02% polyvinylpyrrolidone, 0.02% Ficoll, 0.02% bovine serum albumin), 0.5% sodium dodecyl sulfate (SDS), and 20 μg/ml salmon sperm DNA. The filters were washed twice for 20 min with 2×SSC/10% SDS at room temperature, and for 30 min with 0.2×SSC/10% SDS at 65 C, followed by autoradiography. From about 2×10⁶ recombinants, two positive clones were plaque purified, subcloned into pBluescript SK⁺ vector. PCR screening of the mentioned libraries gave similar results. Complete cDNA inserts were sequenced according to the method of Sanger [13], using a primer walking strategy starting from primers flanking the multiple cloning site of the plasmid. Sequence analysis was performed using Geneworks software system. For homology searches NCBI BLASTx and BLASTn software were used. Several clones were isolated and sequenced. Sequence analysis of one alone revealed a 822 bp cDNA (SEQ ID NO: 8) predicting a protein length of 274 amino acids (SEQ ID NO: 2).

The calculated molecular weight is 29428.02 Dalton and the estimated pi is 9.49. The amino acid composition is as follows:

| Non-polar: | No. | Percent |
|---|---|---|
| A | 34 | 12.36 |
| V | 25 | 9.09 |
| L | 27 | 9.82 |
| I | 13 | 4.73 |
| P | 12 | 4.36 |
| M | 4 | 1.45 |
| F | 18 | 6.55 |
| W | 6 | 2.18 |
| Polar: | No. | Percent |
| G | 27 | 9.82 |
| S | 25 | 9.09 |
| T | 13 | 4.73 |
| C | 2 | 0.73 |
| Y | 9 | 3.27 |
| N | 3 | 1.09 |
| Q | 10 | 3.64 |
| Acidic: | No. | Percent |
| D | 9 | 3.27 |
| E | 8 | 2.91 |
| Basic: | No. | Percent |
| K | 10 | 3.64 |
| R | 13 | 4.73 |
| H | 6 | 2.18 |

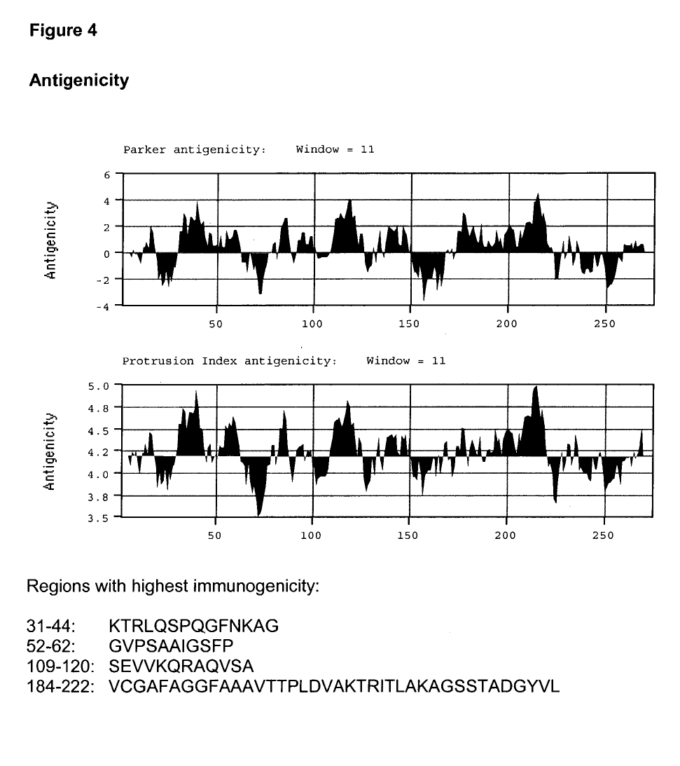

A parker antigenicity plot revealed regions with highest immunogenicity (see FIG. 4):

| | |
|---|---|
| 31-44: | KTRLQSPQGFNKAG |
| 52-62: | GVPSAAIGSFP |
| 109-120: | SEVVKQRAQVSA |
| 184-222: | VCGAFAGGFAAAVTTPLDVAKTRITLAKAGSSTADGYVL |

PSORT analysis shows the likelihood of the localization of the protein in (%) in the following cellular structures:

| | |
|---|---|
| 34.8% | mitochondrial |
| 26.1% | cytoplasm |
| 17.4% | endoplasmatic Reticulum |
| 13% | golgi |
| 4.3% | vaculoar |
| 4.3% | nuclear |

The translated aminoacid sequence of 274 amino acids does share very high homology (>80% identities) with murine proteins AK015954 and AK015299. Beyond this, homologies has been found to be present in “Mitochondrial Carrier Protein Family, Pet8p′ in S. cerevisiae (43% identities).

The proteins of the Pet8p family play an essential role in cellular energy metabolism. Given the sequence homology between TZON7 and “Mitochondrial Carrier Protein family Pet8p”, it is likely that TZON7 is important for growth of activated T cells and useful as a therapeutic marker or may provide novel approaches for monitoring human organ transplantation and leukemia. Means and methods to test the biological activity of the (poly)peptides of the invention, antibodies, antisense constructs and other compounds described in the general description of the invention can be performed as described in the examples of, for example, WO99/11782 and WO01/32614 which herewith are incorporated by reference.

TZON7 may also be useful in the same fashion as human regulatory molecules, including in the manner that mitochondrial carrier protein is described in U.S. Pat. Nos. 5,932,442 and 6,132,973. See also U.S. Pat. No. 6,476,195.

It is also likely that TZON7 may be useful in the control of cell signalling and communication, including the signals that control apoptosis. In this manner, TZON7 may be important in therapies for disorders characterized by disruption of the normal cell cycle, specifically programmed apoptosis or cell death, as in cancer.

Those skilled in the art will recognize, or be able to ascertain using no more than routine experimentation, many equivalents to the specific embodiments of the invention described herein. Such equivalents are intended to be encompassed by the following claims.

The invention will now be further described by the following numbered paragraphs:

1. A polynucleotide encoding a TZON7 polypeptide or a biologically active fragment thereof comprising a nucleic acid molecule selected from the group consisting of

(i) DNA sequences encoding the amino acid sequence depicted in any one of SEQ ID NOS: 2, 4, or 9 to 13;

(ii) the DNA sequence depicted in SEQ ID NO. 1 or 8;

(iii) DNA sequences encoding a fragment or derivative of the protein encoded by the DNA sequence of (i) or (ii);

(iv) DNA sequences the complementary strand of which hybridizes with and which is at least 70% identical to the polynucleotide as defined in any one of (i) to (iii); and

(v) DNA sequences the nucleotide of which is degenerate to the nucleotide sequence of a DNA sequence of any one of (i) to (iv).

2. Use of a nucleic acid molecule of at least 15 nucleotides in length hybridizing specifically with a polynucleotide of claim 1 or with a complementary strand thereof for the detection and/or amplification of a polynucleotide of claim 1 or for repression or targeting of a gene comprising a polynucleotide of claim 1.

3. A vector comprising the polynucleotide of claim 1 or the nucleic acid molecule of claim 2.

4. The vector of claim 3, wherein said polynucleotide or nucleic acid molecule is operably linked to regulatory sequences allowing for the transcription and, optionally, expression of said nucleic acid molecule.

5. A host comprising a polynucleotide of claim 1 or the vector of claim 3 or 4.

6. A method for the production of a TZON7 protein/polypetide or a biologically active fragment thereof comprising:

(a) culturing the host of claim 5 under conditions allowing for the expression of the protein; or

(b) in vitro translation of the polynucleotide of claim 1;

and recovering the protein produced in (a) or (b).

7. A TZON7 protein/polypeptide or a biologically active fragment thereof encoded by the polynucleotide of claim 1 or produced by the method of claim 6.

8. An antibody specifically recognizing the protein of claim 7.

9. A normal cell that has been modified to express the protein of claim 7 or the antibody of claim 8.

10. An antisense construct capable of inhibiting the expression of the polynucleotide of claim 1.

11. A pharmaceutical composition comprising the polynucleotide of claim 1, the nucleic acid molecule as defined in claim 2, a vector of claim 3 or 4, the cell of claim 5, the protein of claim 7, the antibody of claim 8 or the antisense construct of claim 10 and optionally a pharmaceutically acceptable carrier.

12. The pharmaceutical composition of claim 11 for use in cell or organ transplantation, for the treatment of autoimmune, allergic or infectious diseases, or for the treatment of tumors or for the improvement of allograft or xenograft tolerance.

13. A diagnostic composition comprising a polynucleotide of claim 1, the nucleic acid molecule as defined in claim 2, the vector of claim 3 or 4, the cell of claim 5 or 9, the protein of claim 7, or the antibody of claim 8; and optionally, at least one component which is labeled.

14. A method of diagnosing a pathological condition or a susceptibility to a pathological condition in a subject related to a disorder in the immune system comprising:

(a) determining the presence or absence of a mutation in the polynucleotide of claim 1; and

(b) diagnosing a pathological condition or a susceptibility to a pathological condition based on the presence or absence of said mutation:

15. A method of diagnosing a pathological condition or a susceptibility to a pathological condition in a subject related to a disorder in the immune system comprising:

(a) determining the presence or amount of expression of the protein of claim 7 in a biological sample; and

(b) diagnosing a pathological condition or a susceptibility to a pathological condition based on the presence or amount of expression of the protein.

16. A method for identifying a binding partner to a TZON7 protein comprising:

(a) contacting a protein of claim 7 with a compound to be screened; and

(b) determining whether the compound effects an activity of the protein.

17. A method for identifying leukocyte/lymphocyte activating or co-stimulating compounds or for identifying inhibitors of leukocyte activation and stimulation comprising

(a) culturing leukocytes, lymphocytes or monocytes in the presence of the protein of claim 7, the antibody of claim 8, the cell of claim 5 or 9 and, optionally, in the presence of a component capable of providing a detectable signal in response to leucocyte proliferation, with a compound to be screened under conditions permitting interaction of the compound with the (poly)peptide, antibody or cell(s); and

(b) detecting the presence or absence of a signal generated from the interaction of the compound with the cells.

18. A method for the production of a pharmaceutical composition comprising the steps of the method of claim 16 or 17 and formulating and optionally synthesizing the compound identified in step (b) in a pharmaceutically acceptable form.

19. A method for determining the status of an immune response comprising analyzing the presence of the polynucleotide of claim 1 or the protein of claim 7.

20. Use of the polynucleotide of claim 1, the nucleic acid molecule as defined in claim 2, the vector of claim 3 or 4, the protein of claim 7, the antibody of claim 8, the cell of claim 5 or 9, the antisense construct of claim 10 or the compound identified according to the method of any on of claims 14 to 17 for the preparation of a composition for diagnosing or the treatment of acute and chronic diseases, involving T cell activation and Th1 and Th2 immune response, for the treatment of acute and chronic rejection of allo-and xeno organ transplants and bone marrow transplantation, for the treatment of rheumatoid arthritis, lupus erythematodes, multiple sclerosis, encephalitis, vasculitis, diabetes mellitus, pancreatitis, gastritis, thyroiditis, for the treatment of maligne disorders of T, B or NK cells, for the treatment of asthma, lepramatosis, Helicobacter pylori associated gastritis or for the treatment of skin tumors, adrenal tumors or lung tumors, wound healing, growth disorders, inflammatory and/or infectious diseases.

21. Use of a polynucleotide of claim 1, a nucleic acid molecule as defined in claim 2 or the antibody of claim 8 for the detection of leucocyte/lymphocyte activation.

22. Use of claim 20, wherein said leucocyte/lymphocyte is a B cell, T cell, NK cell and/or monocyte.

### REFERENCES

(1) G. R. Crabtree, Contingent genetic regulatory events in T lymphocyte activation, Science 248 (1989) 355-361.

(2) C. H. June, Signal transduction in T-cells, Curr. Opin. Immunol. 3 (1991) 287-293

(3) R. H. Schwartz, Costimulation of T lymphocytes: The role of CD28, CTLA-4, and B7/BB1 in Interleukin-2 production and immunotherapy, Cell 71 (1992), 1065-1068

(4) J. Banchereau, F. Bazan, D. Blanchard, F. Briere, J. Galizzi, C. van Kooten, Y. Liu, F. Rousset, S. Seeland, The CD40 antigen and ist ligand, Annu. Rev. Immunol. 12 (1994) 881-992.

(5) D. J. Lenschow, T. Walunas, J. Bluestone, CD28/B7 system of T-cell costimulation, Annu. Rev. Immunol. 14 (1996) 233-258.

(6) P. Linsley, J. Ledbetter, The role of the CD28 receptor during T-cell responses to antigen, Annu. Rev. Immunol. 11 (1993) 191-212.

(7) A. Kupfer, S. L. Swain, S. J. Singer, The specific direct interaction of helper T-cells and antigen-presenting B cells. II. Reorientation of the microtubule organizing center and reorganization of the membrane-associated cytoskeleton inside the bound helper T-cells, J. Exp. Med. 165 (1987) 1565-1580.

(8) M. V. Parsey, G. K. Lewis, Actin polymerization and pseudopod reorganization accompany anti-CD3-induced growth arrest in Jurkat T-cells, J. Immunol. 151 (1993) 1881-1893.

(9) N. Selliah, W. H. Brooks, T. L. Roszman, Proteolytic cleavage of -actinin by calpain in T-cells stimulated with anti-CD3 monoclonal antibody, J. Immunol. 156 (1996) 3215-3221.

(10) R. Kojima, J. Randall, B. M. Brenner, S. R. Gullans, Osmotic stress protein 94 (Osp94): A new member of the Hsp119/SSE gene subfamily, J. Biol. Chem. 271 (1996) 12327-12332.

(11) J. Sambrook, E. F. Fritsch, T. Maniatis, Molecular Cloning: A Laboratory Manual, Cold Spring Harbour, N.Y., 1989.

(12) A. P. Feinberg, B. Vogelstein, A technique for radiolabeling DNA restriction endonuclease fragment to high specific activity, Anal. Biochem. 132 (1983) 6-13.

(13) F. Sanger, S. Nicklen, A. R. Coulson, DNA sequencing with chain-terminating inhibitors, Proc. Natl. Acad. Sci. USA 74 (1977) 5463-5467.

1. A polynucleotide encoding a TZON7 polypeptide or a biologically active fragment thereof comprising a nucleic acid molecule selected from the group consisting of:
(i) DNA sequences encoding the amino acid sequence depicted in any one of SEQ ID NOS: 2, 4, or 9 to 13;
(ii) the DNA sequence depicted in SEQ ID NO. 1 or 8;
(iii) DNA sequences encoding a fragment or derivative of the protein encoded by the DNA sequence of (i) or (ii);
(iv) DNA sequences the complementary strand of which hybridizes with and which is at least 70% identical to the polynucleotide as defined in any one of (i) to (iii); and
(v) DNA sequences the nucleotide of which is degenerate to the nucleotide sequence of a DNA sequence of any one of (i) to (iv).

2. A nucleic acid molecule of at least 15 nucleotides in length which hybridizes specifically with a polynucleotide of claim 1 or with a complementary strand thereto.

3. A method of detecting and/or amplifying a polynucleotide of claim 1 or for repressing or targeting of a gene comprising a polynucleotide of claim 1, wherein the method comprises using a nucleic acid molecule of at least 15 nucleotides in length which hybridizes specifically with a polynucleotide of claim 1 or with a complementary strand thereof.

4. A vector comprising the polynucleotide of claim 1 or a nucleic acid molecule of at least 15 nucleotides in length which hybridizes specifically with a polynucleotide of claim 1 or with a complementary strand thereto.

5. The vector of claim 4, wherein said polynucleotide is operably linked to regulatory sequences allowing for the transcription and, optionally, expression of said nucleic acid molecule.

6. A host cell comprising:
a polynucleotide of claim 1; or
a vector comprising:
the polynucleotide of claim 1; or
a nucleic acid molecule of at least 15 nucleotides in length which hybridizes specifically with a polynucleotide of claim 1 or with a complementary strand thereto.

7. A method for the production of a TZON7 protein/polypetide or a biologically active fragment thereof comprising culturing the host cell of claim 6 under conditions allowing for the expression of the protein and recovering the protein produced.

8. A method for the production of a TZON7 protein/polypetide or a biologically active fragment thereof comprising in vitro translation of the polynucleotide of claim 1 and recovering the protein produced.

9. A TZON7 protein/polypeptide or a biologically active fragment thereof encoded by the polynucleotide of claim 1 or produced by in vitro translation of the polynucleotide of claim 1 and recovering the protein produced.

10. A TZON7 protein/polypeptide or a biologically active fragment thereof produced by the method of claim 7.

11. An antibody specifically recognizing the protein of claim 9.

12. An antibody specifically recognizing the protein of claim 10.

13. A normal cell that has been modified to express the protein of claim 9.

14. A normal cell that has been modified to express the protein of claim 10.

15. A normal cell that has been modified to express the antibody of claim 11.

16. A normal cell that has been modified to express the antibody of claim 12.

17. An antisense construct capable of inhibiting the expression of the polynucleotide of claim 1.

18. A pharmaceutical composition comprising the polynucleotide of claim 1 and optionally:
a nucleic acid molecule of at least 15 nucleotides in length which hybridizes specifically with a polynucleotide of claim 1 or with a complementary strand thereto;
a vector comprising the polynucleotide of claim 1 or a nucleic acid molecule of at least 15 nucleotides in length which hybridizes specifically with a polynucleotide of claim 1 or with a complementary strand thereto;
a host cell comprising:
a polynucleotide of claim 1; or
a vector comprising:
the polynucleotide of claim 1; or
a nucleic acid molecule of at least 15 nucleotides in length which hybridizes specifically with a polynucleotide of claim 1 or with a complementary strand thereto;
a TZON7 protein/polypeptide or a biologically active fragment thereof encoded by the polynucleotide of claim 1 or produced by in vitro translation of the polynucleotide of claim 1 and recovering the protein produced;
an antibody specifically recognizing the TZON7 protein/polypeptide or a biologically active fragment thereof; or
an antisense construct capable of inhibiting the expression of the polynucleotide of claim 1;
and optionally a pharmaceutically acceptable carrier.

19. The pharmaceutical composition of claim 18 for use in cell or organ transplantation, for the treatment of autoimmune, allergic or infectious diseases, or for the treatment of tumors or for the improvement of allograft or xenograft tolerance.

20. A diagnostic composition comprising at least one of the following:
a polynucleotide of claim 1;
a nucleic acid molecule of at least 15 nucleotides in length which hybridizes specifically with a polynucleotide of claim 1 or with a complementary strand thereto;
a vector comprising the polynucleotide of claim 1 or a nucleic acid molecule of at least 15 nucleotides in length which hybridizes specifically with a polynucleotide of claim 1 or with a complementary strand thereto;
a host cell comprising:
a polynucleotide of claim 1; or
a vector comprising:
the polynucleotide of claim 1; or
a nucleic acid molecule of at least 15 nucleotides in length which hybridizes specifically with a polynucleotide of claim 1 or with a complementary strand thereto;
a TZON7 protein/polypeptide or a biologically active fragment thereof encoded by the polynucleotide of claim 1 or produced by in vitro translation of the polynucleotide of claim 1 and recovering the protein produced; or
an antibody specifically recognizing the TZON7 protein/polypeptide or a biologically active fragment thereof;
and optionally, at least one component which is labeled.

21. A method of diagnosing a pathological condition or a susceptibility to a pathological condition in a subject related to a disorder in the immune system comprising:
(a) determining the presence or absence of a mutation in the polynucleotide of claim 1; and
(b) diagnosing a pathological condition or a susceptibility to a pathological condition based on the presence or absence of said mutation.

22. A method of diagnosing a pathological condition or a susceptibility to a pathological condition in a subject related to a disorder in the immune system comprising:
(a) determining the presence or amount of expression of the protein of claim 9 in a biological sample; and
(b) diagnosing a pathological condition or a susceptibility to a pathological condition based on the presence or amount of expression of the protein.

23. A method of diagnosing a pathological condition or a susceptibility to a pathological condition in a subject related to a disorder in the immune system comprising:
(a) determining the presence or amount of expression of the protein of claim 10 in a biological sample; and
(b) diagnosing a pathological condition or a susceptibility to a pathological condition based on the presence or amount of expression of the protein.

24. A method for identifying a binding partner to a TZON7 protein comprising:
(a) contacting a protein of claim 9 with a compound to be screened; and
(b) determining whether the compound effects an activity of the protein.

25. A method for identifying a binding partner to a TZON7 protein comprising:
(a) contacting a protein of claim 10 with a compound to be screened; and
(b) determining whether the compound effects an activity of the protein.

26. A method for identifying leukocyte/lymphocyte activating or co-stimulating compounds or for identifying inhibitors of leukocyte activation and stimulation comprising:
(a) culturing leukocytes, lymphocytes or monocytes in the presence of one of the following:
a host cell comprising:
a polynucleotide of claim 1; or
a vector comprising:
the polynucleotide of claim 1; or
a nucleic acid molecule of at least 15 nucleotides in length which hybridizes specifically with a polynucleotide of claim 1 or with a complementary strand thereto;
a TZON7 protein/polypeptide or a biologically active fragment thereof encoded by the polynucleotide of claim 1 or produced by in vitro translation of the polynucleotide of claim 1 and recovering the protein produced; or
an antibody specifically recognizing the TZON7 protein/polypeptide or a biologically active fragment thereof;
and, optionally, in the presence of a component capable of providing a detectable signal in response to leucocyte proliferation, with a compound to be screened under conditions permitting interaction of the compound with the (poly)peptide, antibody or cell(s); and
(b) detecting the presence or absence of a signal generated from the interaction of the compound with the cells.

27. A method for the production of a pharmaceutical composition comprising the steps of the method of claim 24 and formulating and optionally synthesizing the compound identified in step (b) in a pharmaceutically acceptable form.

28. A method for the production of a pharmaceutical composition comprising the steps of the method of claim 25 and formulating and optionally synthesizing the compound identified in step (b) in a pharmaceutically acceptable form

29. A method for the production of a pharmaceutical composition comprising the steps of the method of claim 26 and formulating and optionally synthesizing the compound identified in step (b) in a pharmaceutically acceptable form

30. A method for determining the status of an immune response comprising analyzing the presence of:
the polynucleotide of claim 1; or
the TZON7 protein/polypeptide or a biologically active fragment thereof encoded by the polynucleotide of claim 1; or produced by in vitro translation of the polynucleotide of claim 1 and recovering the protein produced; or produced by culturing a host cell comprising:
a polynucleotide of claim 1; or
a vector comprising:
the polynucleotide of claim 1; or
a nucleic acid molecule of at least 15 nucleotides in length which hybridizes specifically with a polynucleotide of claim 1 or with a complementary strand thereto under conditions allowing for the expression of the protein and recovering the protein produced.

31. A method of preparing a composition for diagnosing or the treatment of acute and chronic diseases, involving T cell activation and Th1 and Th2 immune response, for the treatment of acute and chronic rejection of allo-and xeno organ transplants and bone marrow transplantation, for the treatment of rheumatoid arthritis, lupus erythematodes, multiple sclerosis, encephalitis, vasculitis, diabetes mellitus, pancreatitis, gastritis, thyroiditis, for the treatment of maligne disorders of T, B or NK cells, for the treatment of asthma, lepramatosis, Helicobacter pylori associated gastritis or for the treatment of skin tumors, adrenal tumors or lung tumors, wound healing, growth disorders, inflammatory and/or infectious diseases comprising one or more of:
the polynucleotide of claim 1;
a nucleic acid molecule of at least 15 nucleotides in length which hybridizes specifically with a polynucleotide of claim 1 or with a complementary strand thereto;
a vector comprising the polynucleotide of claim 1 or a nucleic acid molecule of at least 15 nucleotides in length which hybridizes specifically with a polynucleotide of claim 1 or with a complementary strand thereto;
a host cell comprising:
a polynucleotide of claim 1; or
a vector comprising:
the polynucleotide of claim 1; or
a nucleic acid molecule of at least 15 nucleotides in length which hybridizes specifically with a polynucleotide of claim 1 or with a complementary strand thereto;
a TZON7 protein/polypeptide or a biologically active fragment thereof encoded by the polynucleotide of claim 1 or produced by in vitro translation of the polynucleotide of claim 1 and recovering the protein produced;
an antibody specifically recognizing the TZON7 protein/polypeptide or a biologically active fragment thereof; and
an antisense construct capable of inhibiting the expression of the polynucleotide of claim 1

32. A method of detecting leucocyte or lymphocyte activation comprising using one or more of:
a polynucleotide of claim 1;
a nucleic acid molecule of at least 15 nucleotides in length which hybridizes specifically with a polynucleotide of claim 1 or with a complementary strand thereto; or
the antibody specifically recognizing a TZON7 protein/polypeptide or a biologically active fragment thereof encoded by the polynucleotide of claim 1 or produced by in vitro translation of the polynucleotide of claim 1 and recovering the protein produced.

33. The method of claim 32, wherein said leucocyte or lymphocyte is a B cell, T cell, NK cell and/or monocyte.
